(19) United States
(12) Patent Application Publication
(10) Pub. No.: US 2021/0290489 A1
(43) Pub. Date: Sep. 23, 2021

(54) IMPROVEMENTS IN OR RELATED TO TRANSDERMAL DELIVERY

(71) Applicant: NDM TECHNOLOGIES LIMITED, Leicestershire (GB)

(72) Inventor: Dewan Fazlul Hoque CHOWDHURY, Leicestershire (GB)

(73) Assignee: NDM TECHNOLOGIES LIMITED, Leicestershire (GB)

(21) Appl. No.: 16/317,393

(22) PCT Filed: Jul. 12, 2017
(86) PCT No.: PCT/GB2017/052043

§ 371 Date: Jan. 11, 2019

(30) Foreign Application Priority Data
Jul. 13, 2016 (GB) 1612171.7

(51) Int. Cl. A61J 1/20

(52) U.S. Cl. CPC A61J 1/2096; A61J 1/2089; A61J 1/2058; A61J 1/2013

(57) ABSTRACT

It is known to deliver drugs through the skin, i.e. transdermally, by using a needle that is used in conjunction with a device.

A device (10) comprises a device body (12) which has an opening (14) for receiving a needle assembly (100) in use. The device also comprises a loading mechanism (16) which is moveable in use to receive the needle assembly (100) via the opening (14) and secure the needle assembly (100) relative to the device body (12). The loading mechanism (16) is configured to receive and secure the needle assembly (100) upon force exerted on the loading mechanism (16) in a single direction (F_{D1}).

This invention relates to a device, a needle assembly for use with the device, a needle assembly device and a container housing the needle assembly.

It is known to deliver drugs through the skin, i.e. transdermally, by using a needle.

An example of how a drug is delivered using a needle is via a pen injector device for the delivery of insulin. Such a device consists of a vial of drug which acts as the barrel of a syringe and a needle assembly including a housing which has a needle tip protruding from both ends of the housing. One needle tip end is designed to puncture a septum in the drug vial and the other needle tip end is designed to pierce the skin of the patient for drug delivery. With this type of device, the needles must be replaced after each dose administered. Some injector devices have the needle pre-loaded within the device and such devices are generally disposable, such as pen type injectors for injecting epinephrine as well as devices for insulin delivery. The needle of such a device is usually protected by a protecting cap that covers the tip of the device. Alternatively, the needles are held in a retracted position within the device housing when not in use such that the needles do not require a separate protecting cap.

For the types of device where the needle is not already pre-loaded, the needle and the injector (or syringe) device are separate components and the needle is manually attached onto the device at the point of use. The needle is usually attached onto the end of the device by either pressing the needle onto the end of a leur slip connection of the device or by rotating the needle to lock it to a leur lock mechanism of the device.

When attaching the needle onto the syringe device, and preferably when also disposing of the needle from the device, it is preferable to do so without a user having to handle any part of the needle. In this way, unwanted needle injuries are prevented as is contamination of the needle and its associated housing.

It is known to cover the needle with a cap so as to prevent needle injuries. The cap is then removed and disposed of prior to using the needle. The problem with using a needle cap is that it is prone to falling off, which can result in the needle and also the associated housing becoming contaminated. Moreover, the needle cap is an additional component which adds to the cost and overall waste of the injector device.

The word device is used herein to describe a system or structure into which a needle is loaded. The word needle is used here to describe one or more needles and associated housing and conduits where present. The term vial is used to describe a reservoir for the storage of a drug or other pharmaceutical agent, biological drug, vaccine, or cosmetic agent, intended for administration into a patient who may be a human or animal subject.

According to an aspect of the invention there is provided a device comprising:
a device body having an opening for receiving a needle assembly in use; and
a loading mechanism moveable in use to receive the needle assembly via the opening and secure the needle assembly relative to the device body, wherein the loading mechanism is configured to receive and secure the needle assembly upon force exerted on the loading mechanism in a single direction.

The loading mechanism being configured to receive and secure the needle assembly upon force exerted on the loading mechanism in a single direction means that the loading mechanism is capable of securing a needle assembly in a straightforward and efficient manner without the need for a user to handle the needle assembly.

This is because the needle assembly could be laying on a flat surface, e.g. a table, and the device can be positioned over the needle assembly and pushed downwards towards the needle assembly, thus resulting in the needle assembly exerting a force onto the loading mechanism in a single direction. Alternatively, the needle assembly could be in an opened container and pushed into the device while the container is lying on a palm of a hand, for example, thus resulting in the needle assembly exerting a force onto the loading mechanism in a single direction. Since such a force would result in both the receiving and securing of the needle assembly in the device, a user does not have to handle the needle assembly.

Meanwhile, the loading mechanism being moveable to receive and secure the needle assembly means that it is the device which moves to achieve the receiving and securing rather than a user having to manipulate the needle assembly to achieve the receiving and securing.

The loading mechanism may be a “stand alone” mechanism which is included in the device. The loading mechanism may instead form part of an existing component belonging to or contained within the device. In the latter regard, the loading mechanism may form part of a vial included within the device.

Optionally the loading mechanism is configured to move in a first direction to receive the needle assembly and in a second direction to secure needle assembly relative to the device body. In some embodiments of the invention, the first and second directions are substantially perpendicular to one another.

In a particular embodiment of the invention the loading mechanism includes a receiving member movable in the first direction from a first position to a second position to receive the needle assembly, and a securing member moveable in the second direction from a first position to a second position to secure the needle assembly relative to the device body.

Having such members arranged in this manner provides an efficient and reliable way of achieving the receiving and securing of the needle assembly.

Preferably the receiving member is configured to prevent movement of the securing member when the receiving and securing members are in the respective first positions, and wherein the receiving member is configured to permit movement of the securing member when the receiving and securing members are in the respective second positions.

The receiving member may include an abutting surface and an aperture and the securing member may include a projection, wherein the projection is configured to abut the abutting surface to prevent movement of the securing member when the receiving and securing members are in the respective first positions, and wherein the aperture receives the projection to permit movement of the securing member when the receiving and securing members are in the respective second positions.

Preferably the receiving member is biased towards a third direction opposite to the first direction.

The receiving member being biased towards a third direction opposite to the first direction means that the receiving member is configured to return to the first position when the securing member is moved from the second position to the first position. Therefore, an additional means for returning the receiving member to the first position is not required.

Preferably the securing member is biased towards the second direction.

The securing member being biased towards the second direction means that the securing member is configured to move to the second position when the receiving member is moved from the first position to the second position. Therefore, an additional means to move the securing member to the second position is not required.

The device may further include an ejection mechanism configured to release the needle assembly in use from the loading mechanism upon a force exerted on the ejection mechanism in a single direction.

Providing such an ejection mechanism means that a user can eject the needle assembly from the device after use without having to handle the needle assembly. Moreover, the ejection mechanism being configured to release the needle assembly upon a force exerted on the ejection mechanism in a single direction means that the needle assembly can be ejected manually with one hand.

Preferably the ejection mechanism is configured to move the securing member from the second position to the first position to permit the receiving member to return from the second position to the first position.

Such an arrangement provides an effective means for releasing the needle assembly. The ejection mechanism may be integrally formed with the securing member.

Optionally, the device body includes a vial receiving portion to receive in use a vial for containing a pharmaceutical composition, the longitudinal axis of the vial receiving portion being perpendicular to the axis of the opening.

It will be understood that the axis of the opening is an axis which passes through the opening in the direction in which the opening permits the passing of an object (i.e. the direction in which the needle assembly can pass into and out of the opening in this case).

Such an arrangement of the vial receiving portion and the opening means that the length of the device is reduced and that the device is flat when it is being used to administer into the skin, i.e. because the needle assembly in use extends from the longitudinal side of the device. Such an arrangement is particularly advantageous when the needle assembly includes a plurality of microneedles which are applied to the skin over an arching pathway.

This arrangement is in contrast to a known injector device wherein the longitudinal axis of the vial receiving portion is parallel to the axis of the opening, i.e. the needle assembly in use extends from the end of the device.

Preferably the device body includes first and second wall portions spaced from one another to define the opening, the first wall portion being configured to reduce the size of the opening upon movement of the loading mechanism to secure the needle assembly relative to the device body.

The first wall portion being so configured minimises the area of the device which is exposed to potential contamination or to skin during application of the device to the patient. It also reduces the chances of needle injuries.

Optionally the first wall portion is configured to move towards the second wall portion upon movement of the securing member from the first position to the second position.

In this way, movement of the securing member to secure the needle assembly relative to the device body also initiates movement of the first wall portion towards the second wall portion so as to reduce the size of the opening. Thus, the opening size is so reduced once the needle assembly is secured.

Optionally the first wall portion is configured to move away from the second wall portion upon movement of the securing member from the second position to the first position.

In this way, movement of the securing member to release the needle assembly from the device body also initiates movement of the first wall portion away from the second wall portion so as to increase the size of the opening and allow the needle assembly to be released from the device.

According to another aspect of the invention there is provided a needle assembly, for use with the device as described hereinabove, comprising:
a needle body;
a primary needle projection extending from a surface of the needle body; and
a vial connector positioned on surface of the needle body, the vial connector being configured to be in fluid communication with a vial in use.

The primary needle projection may include one or more needles, for example a plurality of needles, and in one example may be microneedles.

Preferably the needle projection and the vial connector are located on adjacent surfaces of the needle body.

The needle projection and the vial connector being located on adjacent surfaces of the needle body means that the needle extends from the longitudinal length (i.e. the side) of the device, instead of the end. This means that the device into which the needle assembly is inserted has a compact length and lies flat against the skin when a drug is being administered.

The needle body preferably has an L-shaped cross-section. Such a cross-section permits the needle assembly to interact with the loading mechanism of the device, in particular the receiving and securing members thereof, so as to permit receiving and securing of the needle assembly relative to the device body.

The vial connector may include a secondary needle projection. The secondary needle projection can pierce a vial in use so as to permit fluid communication between the vial and the needle assembly.

According to a further aspect of the invention there is provided a needle device assembly comprising:
a device as described hereinabove; and
a needle assembly as described hereinabove, the needle assembly being insertable and removeable from the device via the opening,
wherein the force exerted upon the loading mechanism in the single direction is provided by the needle assembly.

Preferably the securing member is configured to abut the needle assembly when the securing member is in the second position.

The securing member abutting the needle assembly when the securing member is in the second position provides a means for the needle assembly to be held in position and thus be secured relative to the device body.

Optionally, the device further includes a vial for containing a pharmaceutical composition located within the device body, the vial and the loading mechanism being positioned so that the needle assembly is held between the securing member in the second position and the vial so as to secure the needle assembly relative to the device body.

In this way, the needle assembly is held in a fixed position between the securing member (in the second position) and the vial so as to secure the needle assembly relative to the device body. Moreover, the needle assembly is in an optimum position to be in fluid communication with the vial so as to permit the pharmaceutical composition to be delivered via the needle assembly.

The primary needle projection may extend towards the opening when the needle assembly is secured relative to the device body.

The primary needle projection extending towards the opening in this way means that only one opening in the device is required to receive the needle assembly and permit administration of a pharmaceutical composition.

Optionally the needle assembly, when secured relative to the device body, is configured to rotate to reveal the primary needle projection extending towards the opening.

In this way, the needle projection is hidden prior to use and so reduces the chances of unwanted needle injury and also helps with needle phobia. Such an arrangement is particularly advantageous for the use of microneedles which are applied to the skin over an arching pathway.

The needle assembly preferably wholly lies within the device body when the needle assembly is secured relative to the device body.

The needle assembly, including the primary needle projection, lying wholly within the device body means that the needle projection is completely hidden right up until the needle projection is to enter the skin. This prevents unwanted needle injuries and contamination, and also helps with users who suffer from needle phobia.

Optionally the loading mechanism and the needle body includes mutually engageable locking portions, the mutually engageable locking portions being configured to engage with one another to lock the needle assembly in position relative to the device body.

Such mutually engageable locking portions help further secure the needle assembly relative to the device body so as to prevent the needle assembly dislodging from the device body.

The needle assembly may further include a vial for containing a pharmaceutical composition located within the device body, wherein the loading mechanism forms part of the vial.

According to a further still aspect of the invention there is provided a container comprising a container body and a needle assembly as described hereinabove housed within the container body.

The container body may include a supporting portion configured to hold the needle assembly in a fixed position relative to the container body.

The supporting portion holding the needle assembly in a fixed position makes it easier for a user to push the needle assembly (while it is in the container) into the device.

According to a further aspect of the invention there is provided a method of administering a drug to a patient using a needle device assembly as described hereinabove.

There now follows a brief description of preferred embodiments of the invention, by way of non-limiting examples, with reference being made to the accompanying drawings in which:

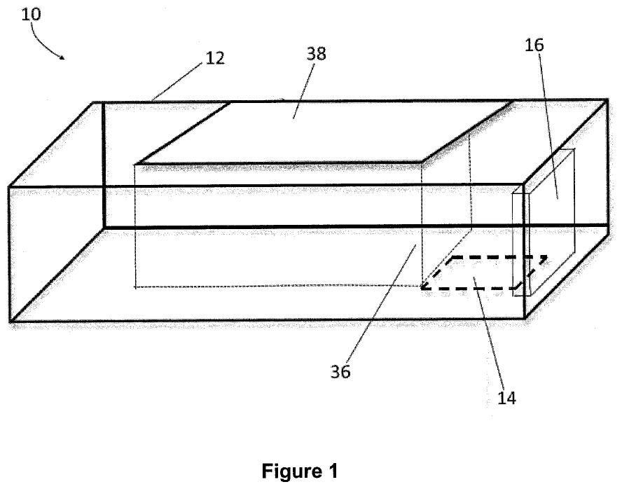

FIG. 1 shows a schematic illustration of a device according to a first embodiment of the invention;

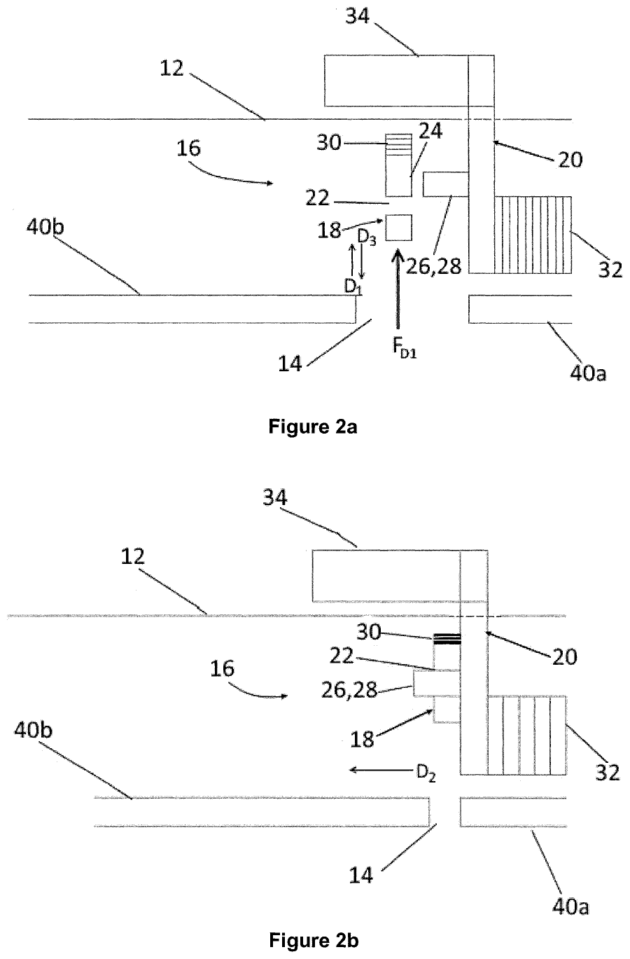

FIG. 2a shows a schematic cross-section of a loading mechanism in a first position of the device shown in FIG. 1;

FIG. 2b shows a schematic cross-section of the loading mechanism of FIG. 2a in a second position;

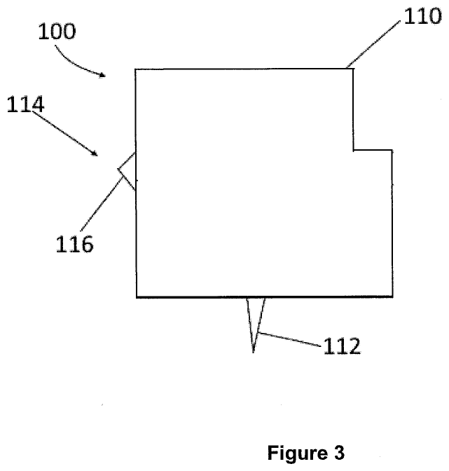

FIG. 3 shows a schematic cross-section of a needle assembly according to a second embodiment of the invention;

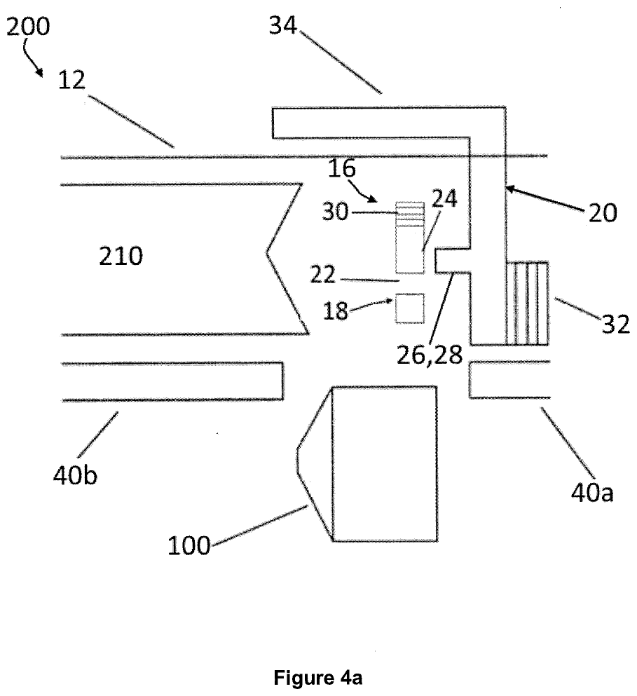

FIG. 4a shows a schematic cross-section of a needle device assembly according to a third embodiment of the invention prior to the needle assembly being inserted;

FIG. 4b shows a schematic cross-section of the needle device assembly of FIG. 4a with the needle assembly inserted;

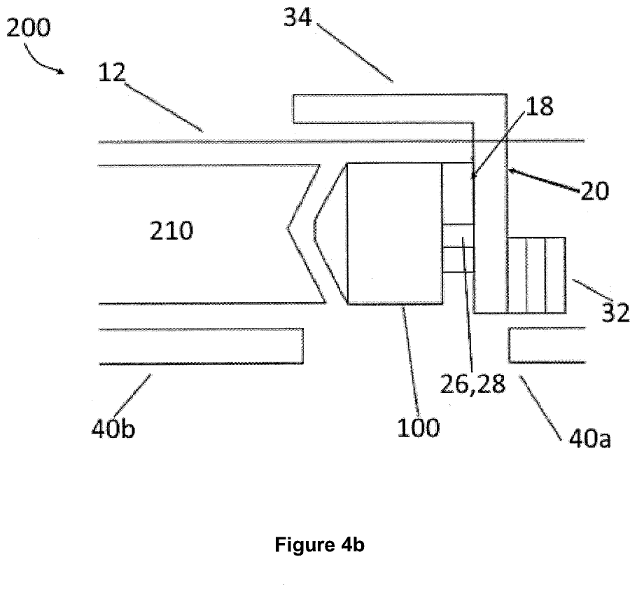

FIG. 5 shows a schematic cross-section of a portion of the loading mechanism of the needle device assembly shown in FIGS. 4a and 4b;

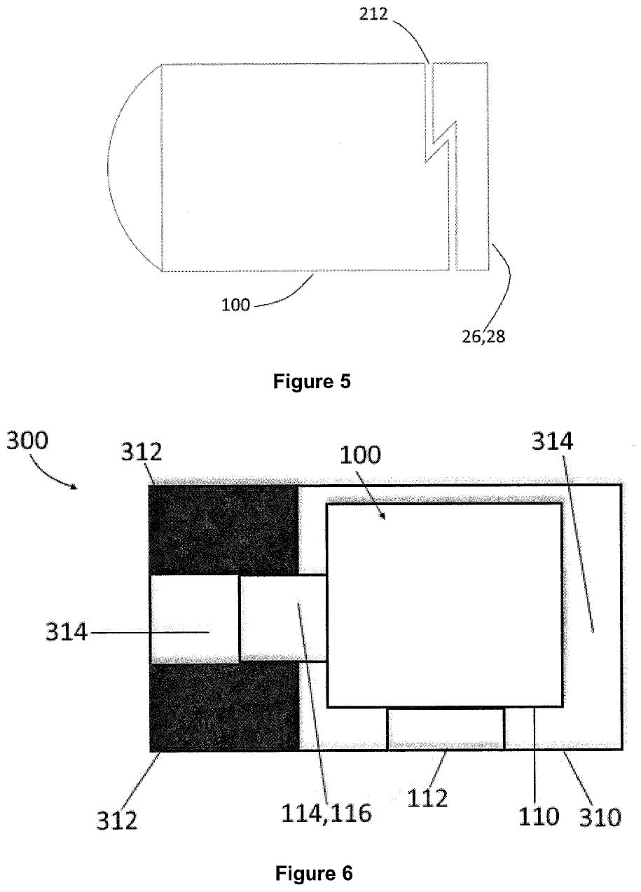

FIG. 6 shows a schematic plan view of a container according to a fourth embodiment of the invention;

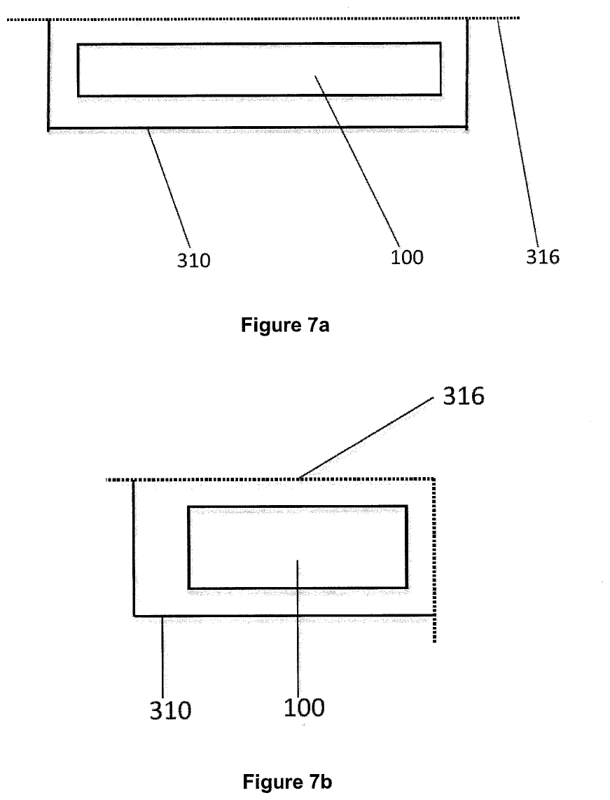

FIG. 7a shows a front cross-section schematic of one configuration of the container of FIG. 6; and

FIG. 7b shows a front cross-section schematic of another configuration of the container of FIG. 6.

A device according to a first embodiment of the invention is shown in FIGS. 1 to 2b and is designated generally by reference numeral 10.

The device 10 includes a device body 12 which has an opening 14 in the base for receiving a needle assembly (not shown in FIGS. 1 to 2b) in use.

The device 10 further includes a loading mechanism 16 (see FIGS. 2a and 2b) which is moveable in use to receive the needle assembly via the opening 14 and secure the needle assembly relative to the device body 12.

The loading mechanism 16 is configured to receive and secure the needle assembly upon force F_{D1} exerted on the loading mechanism 16 in a single direction D₁.

In particular, the loading mechanism 16 is configured to move in a first direction D₁ to receive the needle assembly and in a second direction D₂ to secure the needle assembly relative to the device body 12.

As shown in FIGS. 2a and 2b, the loading mechanism 16 includes a receiving member 18 which is movable in the first direction Di from a first position (FIG. 2a) to a second position (FIG. 2b) to receive the needle assembly. The loading mechanism 16 also includes a securing member 20 which is moveable in the second direction D₂ from a first position (FIG. 2a) to a second position (FIG. 2b) to secure the needle assembly relative to the device body 12.

More specifically, the receiving member 18 is a plate which has an aperture 22 formed therein, wherein the portion of the plate above the aperture 22 provides an abutting surface 24. When viewed in the cross-section of FIGS. 2a and 2b, the plate takes the form of an actuation arm with a portion above the aperture 22 and a portion below the aperture 22. Meanwhile, the securing member 20 includes a projection 26 in the form of a securing pin 28.

The receiving member 18 is biased towards a third direction D₃ opposite to the first direction D₁ via a first spring 30. The first spring 30 is in a rest position in FIG. 2a and a compressed position in FIG. 2b. The securing member 20 is biased towards the second direction D₂ via a second spring 32. The second spring 32 is in a compressed position in FIG. 2a and a rest position in FIG. 2b.

The device 10 also includes an ejection mechanism 34. The ejection mechanism 34 is secured to or integrally formed with the securing member 20 so as to move the securing member 20 from the second position (FIG. 2b) to the first position (FIG. 2a), thus permitting the receiving member 18 to return to the first position (FIG. 2a).

The device 10 also includes a vial receiving portion 36 to receive a vial in use. The device 10 includes a lid 38 on the top side of the device 10 (i.e. opposite the opening 14) for receiving a vial into the vial receiving portion 36. The lid 38 may be on hinges or a sliding mechanism.

Furthermore, the device body 12 includes first and second wall portions 40a, 40b that are spaced from one another to define the opening 14. The first wall portion 40a is configured to move towards and away from the second wall portion 40b so as to reduce and increase the size of the opening 14 as and when the needle assembly is being inserted into or ejected from the device 10. Movement of the first wall portion 40a may occur by producing the wall portion 40a as a moveable sliding mechanism attached to the securing member 20 so they all move in tandem.

A needle assembly 100 for use with the device 10 is shown in FIG. 3.

The needle assembly 100 includes a needle body 110, a primary needle projection 112 extending from a surface of the needle body 110, and a vial connector 114, in the form of a secondary needle projection 116, positioned on a surface of the needle body 110.

The needle projections 112, 116 may be solid metal, plastic, ceramic or glass.

As shown, the primary needle projection 112 and the vial connector 114 are located on adjacent surfaces of the needle body 110. Moreover, the needle body 110 has an L-shaped cross-section.

A needle device assembly according to another embodiment of the invention is shown in FIGS. 4a to 5 and is designated generally by reference numeral 200.

The needle device assembly 200 includes the device 10 and the needle assembly 100 which can be inserted into and out of the opening 14 of the device 10.

The needle device assembly 200 also includes a vial 210 for containing a drug. The vial 210 may be a pre-filled syringe, or a vial with a double septum (one at each end) or some other bespoke reservoir that contains the drug

FIG. 4a shows the loading mechanism 16 in the first position as described above and with the needle assembly 100 ready for insertion. FIG. 4b shows the loading mechanism 16 in the second position as described above and with the needle assembly 100 inserted into and secured relative to the device body 12.

In particular, the needle assembly 100 is secured relative to the device body 12 by the securing pin 28 in the second position trapping the needle assembly 100 between the securing pin 28 and the vial 210.

The vial 210 may interface with the needle assembly 100 to provide a sealed system via a leur lock or leur slip connection or via additional securing means that may involve clamping or compressing the two together.

The primary needle projection 112 may be continuous between the vial 210 and the skin, or there may be a separate conduit between the projection 112 and the vial 210 to bring the two In fluid communication.

In the example shown in FIG. 5, the loading mechanism 16 and the needle body 110 includes mutually engageable locking portions 212 which are configured to engage with one another to lock the needle assembly 100 in position relative to the device body 12.

The locking portions 212 are integral to each of the securing pin 28 and the needle body 110 to form an interlocking mechanism. This interlocking mechanism is designed to ensure the needle assembly 100 can be retracted from within the device body 12 prior to its disconnection and disposal from the device 10. This would occur whereby the movement of the ejection mechanism 34 would lead to the needle assembly 100 also moving towards the first (i.e. rest) position of the loading mechanism 16, and the interlocking mechanism ensures the needle assembly 100 moves with the securing pin 38. The needle assembly 100 can then be removed from the opening 14 of the device 10 such that the needle assembly is ejected out of the device 10 without any manual handling of the needle assembly 100.

FIG. 6 shows a container 300 which includes a container body 310 and the needle assembly 100 housed therein.

The container body 310 includes a supporting portion 312 which is configured to hold the needle assembly 100 in a fixed position relative to the container body 310. This facilitates the location of the needle assembly 100 into the device 10.

The supporting portion 312 may include support structures (not shown) which can be constructed from plastic, paper, foam or other suitable materials that may also be collapsible under exertion. The support structures may be molded as part of the container 300 or may be a secondary material secured to the container 300.

The support structures could for example be the first surface upon which the device 10 is positioned followed by the exertion of pressure which collapses or compresses the support structures allowing the device 10 to sink into the primary packaging void spaces 314 (see below) and engage with the needle assembly 100 to locate it within the device 10. The support structures may be reversibly collapsible, such as a foam material.

As can be seen, the primary needle projection 112 and the secondary needle projection 114 lie adjacent to one another, i.e. their axes are perpendicular to one another.

Void space 314 within the container 300 is present to provide an area for the device 10 to enter into and locate the needle assembly 100 onto the device 10. It follows that the container 300 may therefore have a larger footprint/geometry relative to the part of the device 10 where the needle assembly 100 is to be located and attached, such that it can be completely inserted inside the container 300 utilizing the voids 314.

FIG. 7a shows the container 300 with a sealing membrane 316.

The container 300 may be any suitably sized and shaped container made from a suitable material and construction to provide sufficient rigidity in storage and transit to prevent the projections 112, 114 of the needle assembly 100 from piercing the packaging 300. The container 300 is described as the container in which the needle assembly 100 is directly in contact with, or able to come in to direct contact with its internal surfaces.

The container 300 may be any type of packaging consisting of a high density or high integrity material such as a rigid plastic such as polypropylene, polyester, polystyrene, etc., and a membrane or envelope produced from laminated paper and/or polymer or metal foil, that can be readily peeled off or removed to access the needle assembly 100. The membrane or envelope may be adhered using a heat seal, or a medical adhesive, or using sonic welding. The peel-off envelope/seal may be on one plane or two adjoining planes to allow the needle assembly 100 to be located onto the device 100 horizontally or vertically. The container 300 may take the form of a blister pack.

FIG. 7b shows the container 300 with the sealing membrane 316 at the top and extending over one of the edges. It will be appreciated that this is one of several possible configurations of the container 300 and the sealing membrane 316. The sealing envelope may also be re-sealable for subsequent storage of used needle assemblies 100.

In use, the device 10 is placed firmly on top of the needle assembly 100 (which may be in the container 300 as described above). The needle assembly 100 can be lying flat on a surface such as a table or in the palm of a hand (particularly if it is in the container 300).

The needle assembly 100 engages with the loading mechanism 16 and in particular exerts a force F_{D1} in the first direction D₁ onto the receiving member 18 which is compressed via the first spring 30. As the receiving member 18 is pushed in the first direction D₁, it reaches a position in which the securing pin 28 can move laterally through the aperture 22 of the receiving member 18. The securing pin 28 pushes the needle assembly 100 into a position within the device 10 where it is engaged with the vial 210.

In the case of a leur slip connection, the vial 210 and the needle assembly 100 will engage with adequate force, provided by the securing pin 28, to ensure a watertight seal between the two. In the case of a leur lock mechanism, either the needle assembly 100 or the vial 210 may be rotated to lock the two together, either manually or by use of a suitably mounted cog to turn the vial 210 or the needle assembly 100, using corresponding engagement mechanism/teeth on the vial 210 or needle assembly outer surface.

In an embodiment (not shown) where the needle assembly 100 is positioned horizontally along the longitudinal axis of the vial 210, as is the case for traditional injection devices, a suitable sized orifice is positioned at the end of the device 10 to enable the primary needle projection 112 to protrude at the time of actuation. An actuation mechanism would be included to allow the vial 210 and primary needle projection 112 to be moved vertically down toward the skin. A spring loaded retraction mechanism as used in some pen injectors may be employed to retract the needle projection/vial away from the skin after the administration of the contents of the vial 210.

In the embodiment shown in the drawings, the primary needle projection 112 is administered from the side of the vial 210, perpendicular to the longitudinal axis of the vial 210. The vial 210 in this case would be applied horizontally along the plane of the skin. The primary needle projection 112 may be pushed directly into the skin, or the needle projection 112 may start at a rest position where the tip of the needle projection 112 is not facing the skin, and the vial/needle projection are rolled over the skin to cause the needle projection 112 to penetrate the skin. This would then be followed by injection of the contents of the vial 210 by applying pressure to a suitably mounted piston on the vial 210. In this case there is not a need for an opening at the end of the device 10, and instead the opening 14 on the device 10 used for loading the needle assembly 100 is also used to bring the needle projection 112 into contact with and to pierce the skin.

To eject the needle assembly 100 from the device 10 once the drug has been administered, the ejection mechanism 34 is pushed laterally so as to move the securing pin 28, via the second spring 32, back to the first position. In this position, the securing pin 28 is no longer through the aperture 22 of the receiving member 18, and so the receiving member 18 is free to also move back to the first position via the first spring 30. By doing so, the receiving member 18 pushes the needle assembly 100 out of the device 10 and into the original container 300 which may be resealed for disposal purposes. This avoids the need for users to carry any separate packaging for disposal of the needle assembly 100.

In the case of a leur lock connection the vial 210 is decoupled from the needle assembly 100 by rotating it using a manual or automated means, or using the locking means in the reverse direction. In the case of a leur slip engagement between the vial 210 and needle assembly 100, the needle assembly 100 and/or vial 210 will be pushed away from each other. In order to ensure the ejection mechanism 34 is able to move the otherwise freestanding needle, there is an engagement means (not shown) between the needle assembly 100 and the area of the securing pin 28 that allows the needle assembly 100 to be moved in the horizontal direction but not prevent the needle assembly 100 from being pushed out in the vertical direction for disposal once it is positioned back over the opening 14.

All of the above described mechanisms may be fully automated using suitable cogs, motors, gears, linear actuators, and suitable energy sources, whether mechanical energy sources, or electrochemical or battery powered. The precise mechanisms are not discussed further as these will be obvious to a person skilled in the art.

In a further embodiment the needle assembly may only locate in the device in a fixed vertical position and the vial is then moved to allow fluid communication between the vial and the needle assembly. In a further embodiment there may be a further conduit or channel with associated housing between the needle assembly and the vial. The purpose of this may be to facilitate the engagement between the vial and the needle assembly, for example it may be preferable for the needle assembly to engage with the vial via a leur slip mechanism. An intermediate conduit section consisting of a needle at one tip to puncture the septum within the vial, with a leur lock or leur slip mechanism for securely engaging with the vial, and at the opposite end there may be a leur slip mechanism to engage with the needle.

It will be appreciated that the mechanisms discussed above have been discussed in the context of manual operation, but could all be equally automated using appropriate electro-mechanical actuation means.

1. A device comprising:
a device body having an opening for receiving a needle assembly in use; and
a loading mechanism moveable in use to receive the needle assembly via the opening and secure the needle assembly relative to the device body, wherein the loading mechanism is configured to receive and secure the needle assembly upon force exerted on the loading mechanism in a single direction.

2. A device according to claim 1 wherein the loading mechanism is configured to move in a first direction to receive the needle assembly and in a second direction to secure needle assembly relative to the device body.

3. A device according to claim 2 wherein the loading mechanism includes a receiving member movable in the first direction from a first position to a second position to receive the needle assembly, and a securing member moveable in the second direction from a first position to a second position to secure the needle assembly relative to the device body.

4. A device according to claim 3 wherein the receiving member is configured to prevent movement of the securing member when the receiving and securing members are in the respective first positions, and wherein the receiving member is configured to permit movement of the securing member when the receiving and securing members are in the respective second positions.

5. A device according to claim 3 or claim 4 wherein the receiving member includes an abutting surface and an aperture and the securing member includes a projection, wherein the projection is configured to abut the abutting surface to prevent movement of the securing member when the receiving and securing members are in the respective first positions, and wherein the aperture receives the projection to permit movement of the securing member when the receiving and securing members are in the respective second positions.

6. A device according to any one of claims 3 to 5 wherein the receiving member is biased towards a third direction opposite to the first direction.

7. A device according to any one of claims 3 to 6 wherein the securing member is biased towards the second direction.

8. A device according to any preceding claim further including an ejection mechanism configured to release the needle assembly in use from the loading mechanism upon a force exerted on the ejection mechanism in a single direction.

9. A device according to claim 8 when dependent on any one of claims 3 to 7 wherein the ejection mechanism is configured to move the securing member from the second position to the first position to permit the receiving member to return from the second position to the first position.

10. A device according to any preceding claim wherein the device body includes a vial receiving portion to receive in use a vial for containing a pharmaceutical composition, the longitudinal axis of the vial receiving portion being perpendicular to the axis of the opening.

11. A device according to any preceding claim wherein the device body includes first and second wall portions spaced from one another to define the opening, the first wall portion being configured to reduce the size of the opening upon movement of the loading mechanism to secure the needle assembly relative to the device body.

12. A device according to claim 11 when dependent on any one of claims 3 to 7 wherein the first wall portion is configured to move towards the second wall portion upon movement of the securing member from the first position to the second position.

13. A device according to claim 11 or claim 12 when dependent on claim 9 wherein the first wall portion is configured to move away from the second wall portion upon movement of the securing member from the second position to the first position.

14. A needle assembly, for use with the device according to any one of claims 1 to 13, comprising:
a needle body;
a primary needle projection extending from a surface of the needle body; and
a vial connector positioned on surface of the needle body, the vial connector being configured to be in fluid communication with a vial in use.

15. A needle assembly according to claim 14 wherein the primary needle projection and the vial connector are located on adjacent surfaces of the needle body.

16. A needle assembly according to claim 14 or claim 15 wherein the needle body has an L-shaped cross-section.

17. A needle assembly according to any one of claims 14 to 16 wherein the vial connector includes a secondary needle projection.

18. A needle device assembly comprising:
a device according to any one of claims 1 to 13; and
a needle assembly according to any one of claims 14 to 17, the needle assembly being insertable and removeable from the device via the opening,
wherein the force exerted upon the loading mechanism in the single direction is provided by the needle assembly.

19. A needle device assembly according to claim 18 when dependent on any one of claims 3 to 7 wherein the securing member is configured to abut the needle assembly when the securing member is in the second position.

20. A needle device assembly according to claim 19 further including a vial for containing a pharmaceutical composition located within the device body, the vial and the loading mechanism being positioned so that the needle assembly is held between the securing member in the second position and the vial so as to secure the needle assembly relative to the device body.

21. A needle device assembly according to any one of claims 18 to 20 wherein the primary needle projection extends towards the opening when the needle assembly is secured relative to the device body.

22. A needle device assembly according to any one of claims 18 to 21 wherein the needle assembly, when secured relative to the device body, is configured to rotate to reveal the primary needle projection extending towards the opening.

23. A needle device assembly according to any one of claims 18 to 22 wherein the needle assembly wholly lies within the device body when the needle assembly is secured relative to the device body.

24. A needle device assembly according to any one of claims 18 to 23 wherein the loading mechanism and the needle body includes mutually engageable locking portions, the mutually engageable locking portions being configured to engage with one another to lock the needle assembly in position relative to the device body.

25. A needle device assembly according to claim 18 further including a vial for containing a pharmaceutical composition located within the device body, wherein the loading mechanism forms part of the vial.

26. A container comprising a container body and a needle assembly according to any one of claims 14 to 17 housed within the container body.

27. A container according to claim 26 wherein the container body includes a supporting portion configured to hold the needle assembly in a fixed position relative to the container body.

28. A method of administering a drug to a patient using a needle device assembly according to any one of claims 18 to 25.

29. A device generally as herein described with reference to and/or as illustrated in FIGS. 1 to 2b.

30. A needle assembly generally as herein described with reference to and/or as illustrated in FIG. 3.

31. A needle device assembly generally as herein described with reference to and/or as illustrated in FIGS. 4a to 5.

32. A container generally as herein described with reference to and/or as illustrated in FIGS. 6 to 7b.
